(19) United States
(12) Patent Application Publication
(10) Pub. No.: US 2024/0100149 A1
(43) Pub. Date: Mar. 28, 2024

(54) SARS-COV-2 CONSTRUCTS, VACCINES, AND METHODS

(71) Applicant: THE HOSPITAL FOR SICK CHILDREN, Toronto (CA)

(72) Inventors: Jean-Philippe JULIEN, Toronto (CA); Audrey KASSARDJIAN, Toronto (CA); Brian BARBER, Toronto (CA)

(21) Appl. No.: 18/251,963

(22) PCT Filed: Nov. 5, 2021
(86) PCT No.: PCT/CA2021/051581

§ 371 Date: May 5, 2023

Related U.S. Application Data
(60) Provisional application No. 63/110,881, filed on Nov. 6, 2020.

(51) Int. Cl. A61K 39/215; C07K 14/005; C07K 16/28

(52) U.S. Cl. CPC A61K 39/215; C07K 14/005; C07K 16/2833; A61K 2039/6056; C07K 2319/33

(57) ABSTRACT

Described herein is an anti-class II MHC antibody fused to a SARS-CoV-2 antigen. Also described is a vaccine comprising the antibody and methods for treating and/or preventing SARS-CoV-2, wherein the methods comprise administering the antibody to a subject in need thereof. In typical aspects, the vaccine is free of an adjuvant.

### STATEMENT REGARDING ELECTRONIC FILING OF A SEQUENCE LISTING

A Sequence Listing in ASCII text format, submitted under 37 C.F.R. § 1.821, entitled 1360-42_ST25.txt, 45,039 bytes in size, generated on Nov. 12, 2023, is provided in lieu of a paper copy. This Sequence Listing is hereby incorporated herein by reference into the specification for its disclosures.

### FIELD

The present invention relates to antibodies. In particular, the present invention relates to antibodies, vaccines comprising the antibodies, and related compositions and methods.

### BACKGROUND

It is now generally accepted in the field of immunology that in order for an injected non-self, protein antigen to induce a strong IgG response in mammals, it needs to reach two different classes of cells; specifically, antigen presenting cells (APCs), and B-cells specific for epitopes on the injected antigen₁. Both of these cell types share a key cell surface gene product involved in the development of a strong antibody response, namely class II major histocompatibility (MHC) molecules. Injected antigen needs to be taken up by APCs in the local draining lymph nodes, “processed” (i.e. proteolytically degraded) intracellularly by these cells, with resulting specific antigen fragments being “presented” by binding to class II MHC molecules displayed on the cell surface. At the same time, antigen needs to be recognized by epitope-specific binding to the antigen receptor (i.e. cell surface immunoglobulin), inducing the earliest stage of activation in this B-cell. B-cell receptor engagement with antigen also leads to antigen internalization, as well as proteolytic processing and cell surface presentation of specific antigen fragments on the class II MHC molecules expressed on the antigen-reactive B-cell. As these cellular processes proceed, the class II MHC bound antigenic fragments induce a T-helper cell response in the CD4 class of T-cells. These activated T-cells, which recognize the same antigenic fragment on the class II MHC molecules of antigen reactive B-cells, provide the positive regulatory stimulus to drive maturation (including immunoglobulin class switch) of the antigen-reactive B-cells, such that IgG secreting B-cells (eventually plasma cells) and B-cell memory cells are established.

It is generally recognized that the process described above does not happen efficiently, and in most cases not at all, if a soluble, non-aggregated foreign protein antigen is injected in saline. As such, most vaccines based on purified, pathogen-specific, foreign protein antigens (often called subunit vaccines) are mixed with an immunostimulatory substance (generally called an adjuvant) prior to injection into an animal or a human for the purposes of inducing an anti-pathogen immune response. The adjuvant in a subunit vaccine of this sort is chosen to be immunostimulatory in some particular way, and is thought particularly to promote the antigen uptake, processing and presentation known to be required for the induction of T-helper responses. Unfortunately, many, if not most, of the adjuvants which have been advanced for human vaccine development over the past many decades have proven to be unacceptable in terms of the inflammatory responses that they induce (reviewed by Petrovsky (2015)²). Thus, subunit vaccine development, based on purified pathogen proteins, has been significantly curtailed by the lack of acceptable adjuvants (as judged by the regulatory authorities) for licensed human vaccines.

In an effort to circumvent this considerable roadblock in the development of new subunit vaccines for use in humans, Barber and his colleagues developed an adjuvant-independent approach to vaccine design. As first published in 1987³, Carayanniotis and Barber described the immunotargeting approach to adjuvant-independent vaccine design. Recognizing that class II MHC molecules represent cell surface molecules expressed on both APCs and B-cells, the two types of cells that foreign antigen needs to reach in order to elicit a strong IgG response to the antigen, it was reasoned that coupling antigen to a monoclonal antibody (mAb) specific for class II MHC molecules could circumvent the need for adjuvant. In fact, their results demonstrated that when the foreign protein antigen was physically coupled to the anti-class II MHC mAb, it was able to induce an adjuvant-independent IgG response to the antigen. They also demonstrated that the response was strictly dependent the upon recognition of class II MHC by the anti-class II MHC mAb, and on coupling of the foreign protein antigen to the class II MHC-specific mAb. They called this the immunotargeting approach to subunit vaccine design, and it became a new adjuvant-independent option for the creation of human vaccines.

The utilization of this subunit vaccine technology was extended from the original work in mice to demonstrate that it could also be used to elicit adaptive immune responses in other species including rabbits and ferrets (reviewed by Barber (1997)⁴). Others have also reviewed the more recent work involving the immunotargeting approach to vaccine design^{5,6}.

In a separate approach to enhancing antibody responses to defined antigens, several groups have explored the use of amino acid sequences with the ability to bind to a wide variety of class II MHC molecules and elicit T-helper cell responses. Efforts in this regard have been reviewed by Knutson and Disis (2005)⁷. The specific amino acid sequences which have emerged from these studies are referred to as universal T-helper cell determinants. These include a non-natural 13 amino acid sequence, designated PADRE⁸, and another, designated TpD, which is a 32 amino acid fusion of sequences derived from tetanus toxin and diphtheria toxin, with a short intervening linker sequence⁹. Generally, these sequences are admixed with adjuvants and defined antigens (peptides or proteins) in an effort to elicit more potent responses. Sometimes they enhance immune responses, and sometimes they do not.

The ongoing Coronavirus Disease 2019 (COVID-19) pandemic, caused by the coronavirus designated SARS-CoV-2, has already (by October 2020) infected more than 35 million people, resulting in greater than 1 million deaths worldwide. A safe and effective vaccine which either prevents COVID-19, or substantially blunts its severity, is urgently required.

### SUMMARY OF THE INVENTION

In accordance with an aspect, there is provided an anti-class II MHC antibody fused to a SARS-CoV-2 antigen.

In an aspect, the antibody is an anti-HLA-DR antibody.

In an aspect, the antibody is a broadly reactive anti-HLA-DR antibody.

In an aspect, the antibody is an IgG, scFv, Fab′, Fab, F(ab′)₂, or scFab.

In an aspect, the antibody is an IgG.

In an aspect, the antibody is a monoclonal antibody.

In an aspect, the antibody has a 44H10 specificity.

In an aspect, the antibody is chimeric.

In an aspect, the antibody is a human/mouse chimeric antibody.

In an aspect, the antibody is humanized or human.

In an aspect, the SARS-CoV-2 antigen is a spike protein antigen or a nucleocapsid antigen.

In an aspect, the SARS-CoV-2 antigen is an S1 antigen or an S2 antigen.

In an aspect, the SARS-CoV-2 antigen is an RBD antigen.

In an aspect, the SARS-CoV-2 antigen is fused to a heavy chain of the antibody.

In an aspect, the SARS-CoV-2 antigen is fused at the C-terminus of the heavy chain.

In an aspect, the SARS-CoV-2 antigen is fused to a light chain of the antibody.

In an aspect, the SARS-CoV-2 antigen is fused at the C-terminus of the light chain.

In an aspect, the antibody comprises two heavy chains and/or two light chains and comprising a plurality of SARS-CoV-2 antigens, either the same or different, each fused to a different heavy chain and/or light chain.

In an aspect, the antibody comprises two or more RBD SARS-CoV-2 antigens, each fused to a respective heavy chain or light chain, wherein the RBD SARS-CoV-2 antigens are independently the same or different.

In an aspect, the antibody three or four RBD SARS-CoV-2 antigens, each fused to a respective heavy chain or light chain, wherein the RBD antigens are independently the same or different.

In an aspect, the antibody comprises four RBD SARS-CoV-2 antigens, one at each heavy and light chain.

In an aspect, the antibody comprises two RBD SARS-CoV-2 antigens, each fused to a respective heavy chain.

In an aspect, the antibody further comprises a linker between the antibody and the SARS-CoV-2 antigen.

In an aspect, the linker is a GS repeat linker, such as a GGS×2 linker (SEQ ID NO:16) or GGGGS×2 linker.

In an aspect, the antibody further comprises a universal T-helper determinant.

In an aspect, the universal T-helper determinant comprises PADRE and/or TpD.

In an aspect, the universal T-helper determinant is fused to a heavy chain of the antibody.

In an aspect, the universal T-helper determinant is fused at the C-terminus of the heavy chain.

In an aspect, the universal T-helper determinant is fused to a light chain of the antibody.

In an aspect, the universal T-helper determinant is fused at the C-terminus of the light chain.

In an aspect, the antibody comprises two heavy chains and/or two light chains and a plurality of universal T-helper determinants, either the same or different, each fused to a different heavy chain and/or light chain.

In an aspect, the antibody comprises two universal T-helper determinants, each fused to a respective heavy chain or light chain.

In an aspect, the antibody comprises two universal T-helper determinants, each fused to a respective light chain.

In an aspect, the antibody further comprises a linker between the antibody and the universal T-helper determinant.

In an aspect, the linker is a GS repeat linker, such as a GGS×2 linker (SEQ ID NO:16) or GGGGS×2 linker (SEQ ID NO:17).

In an aspect, the antibody is for use in combination with a vaccine against tetanus and/or diphtheria toxoids.

In an aspect, the antibody comprises a polypeptide sequence having at least 70% sequence identity to any one or more of:

1. Chimeric H chain + RBD:
(SEQ ID NO: 1)
MALLVLFLSLAAFPSCGVLSQVQLKESGPGLVAPSQSLSITCTVSGFSLTSYGVHWVRQPPGKGLE
WLGVIWAGGSINYNSALMSRLSISKDNFKSQVFLKMSSLQTDDTAMYYCARAYGDYVHYAMDYWGQ
GTSVTASSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLS
PGKGGGGSGGGGSRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNF*
Signal peptide
44H10 VH
Human CH (lgG1)
GGGGSx2 linker (SEQ ID NO: 17)
RBD (319-542)
2. Chimeric L chain + RBD:
(SEQ ID NO: 2)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGGGSGGGGSRVQPTESIVRFPNITNLCPFG
EVFNATRFASVYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPFERDISTEIYQA
GSTPCNGVEGFNCYFPLQSYGFQPTNGVGYQPYRVVVLSFELLHAPATVCGPKKSTNLVKNKCVNF*
Signal peptide
44H10 V_{K}
Human C_{K}
GGGGSx2 linker (SEQ ID NO: 17)
RBD (319-542)
3. Chimeric L chain + TpD:
(SEQ ID NO: 3)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGSGGSILMQYIKANSKFIGIPMGLPQSIALSSL
MVAQ*
Signal peptide
44H10 V_{K}
Human C_{K}
GGSx2 linker (SEQ ID NO: 16)
TpD
4. Chimeric L chain + PADRE:
(SEQ ID NO: 12)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGSGGSAKFVAAWTLKAAA*
Signal peptide
44H10 V_{K}
Human C_{K}
GGSx2 linker (SEQ ID NO: 16)
PADRE

In an aspect, the antibody comprises at least one heavy chain and at least one light chain of 1, 2, 3, and 4, in any combination.

In an aspect, the antibody comprises two heavy chains and two light chains of 1, 2, 3, and 4, in any combination.

In an aspect, the antibody consists of two heavy chains and two light chains of 1, 2, 3, and 4, in any combination.

In accordance with an aspect, there is provided a polynucleotide encoding the antibody described herein.

In accordance with an aspect, there is provided a vaccine comprising the polynucleotide described herein.

In accordance with an aspect, there is provided a vector comprising the polynucleotide described herein.

In accordance with an aspect, there is provided a host cell comprising the vector described herein.

In accordance with an aspect, there is provided a SARS-CoV-2 vaccine comprising the antibody described herein.

In an aspect, the SARS-CoV-2 vaccine is free of an adjuvant.

In accordance with an aspect, there is provided a method of immunizing a subject against SARS-CoV-2, the method comprising administering the SARS-CoV-2 vaccine described herein to the subject.

In accordance with an aspect, there is provided a method of treating and/or preventing SARS-CoV-2 in a subject, the method comprising administering the SARS-CoV-2 vaccine described herein to the subject.

In an aspect, the method further comprises administering a vaccine against tetanus and/or diphtheria toxoids to the subject.

In an aspect, the vaccine against tetanus and/or diphtheria toxoids is administered prior to administering the SARS-CoV-2 vaccine.

In an aspect, the vaccine against tetanus and/or diphtheria toxoids is administered to the subject prior to the SARS-CoV-2 vaccine, such as one more days, weeks, months, or years prior to the SARS-CoV-2 vaccine, such as about one month prior to the SARS-CoV-2 vaccine.

In an aspect, the SARS-CoV-2 vaccine is administered without an adjuvant.

In an aspect, the SARS-CoV-2 vaccine is administered as a purified protein without an adjuvant.

In accordance with an aspect, there is provided a use of the SARS-CoV-2 vaccine described herein for immunizing a subject against SARS-CoV-2.

In accordance with an aspect, there is provided a use of the SARS-CoV-2 vaccine described herein for treating and/or preventing SARS-CoV-2.

In an aspect, the use further comprising use of a vaccine against tetanus and/or diphtheria toxoids.

In an aspect, the vaccine against tetanus and/or diphtheria toxoids is for use prior to the SARS-CoV-2 vaccine, such as one more days, weeks, months, or years prior to the SARS-CoV-2 vaccine, such as about one month prior to the SARS-CoV-2 vaccine.

In an aspect, the SARS-CoV-2 vaccine is for use without an adjuvant.

In an aspect, the SARS-CoV-2 vaccine is for use as a purified protein without an adjuvant.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows a schematic representation of the chimeric human IgG1 44H10 anti-HLA-DR antibody and four different mAbs (0, 1, 2 and 3) based on the immunotargeting vector described herein.

FIG. 2A shows the DNA sequence (SEQ ID NO:5) and derived amino acid sequence (SEQ ID NO:4) of the variable region of the murine 44H10 mAb heavy chain (VH) and human IgG1 constant region, used in the expression of the chimeric human IgG1 antibody with 44H10 specificity (Chi-44H10). Each feature of the sequence is identified by annotated boxes.

FIG. 2B shows the DNA sequence (SEQ ID NO:7) and derived amino acid sequence (SEQ ID NO:6) of the variable region of the murine 44H10 mAb light chain (VL) and human kappa constant region, used in the expression of Chi-44H10 and mAb 0. Each feature of the sequence is identified by annotated boxes.

FIG. 2C shows the DNA sequence (SEQ ID NO:9) and derived amino acid sequence (SEQ ID NO:8) of the chimeric mouse/human IgG1 heavy chain used in the expression of mAbs 0, 1, 2, and 3, with the SARS-CoV-2 Spike protein RBD linked to the C-terminus of the heavy chain by a short linker peptide. Each feature of the sequence is identified by annotated boxes.

FIG. 2D shows the DNA sequence (SEQ ID NO:11) and derived amino acid sequence (SEQ ID NO:10) of the chimeric mouse/human kappa (κ) light chain used in the expression of mAb 1, with the T helper determinant TpD linked to the C-terminus of the light chain by a short linker peptide. Each feature of the sequence is identified by annotated boxes.

FIG. 2E shows the DNA sequence (SEQ ID NO:13) and derived amino acid sequence (SEQ ID NO:12) of the chimeric mouse/human kappa (κ) light chain used in the expression of mAb 2, with the T helper determinant PADRE linked to the C-terminus of the light chain by a short linker peptide. Each feature of the sequence is identified by annotated boxes.

FIG. 2F shows the DNA sequence (SEQ ID NO:15) and derived amino acid sequence (SEQ ID NO:14) of the chimeric mouse/human kappa (κ) light chain used in the expression of mAb 3, with the SARS-CoV-2 Spike protein RBD linked to the C-terminus of the light chain by a short linker peptide. Each feature of the sequence is identified by annotated boxes.

FIG. 3 shows representative plasmid maps of the pcDNA3.4 TOPO vector used to express the chimeric 44H10 heavy and light chains in the FreeStyle 293-F cell line. Segments of the plasmids shown in red correspond to the DNA encoding each chain of the chimeric 44H10 antibody. Heavy and light chains of the immunotargeting mAbs were expressed in the same expression vector.

FIG. 4A shows the elution profiles of Chi-44H10 and immunotargeting mAbs purified by protein A chromatography.

FIG. 4B depicts Coomassie Blue-stained SDS-PAGE 4-20% gradient gels. Each purified mAb was run on the gels under non-reducing (NR) and reducing (R) conditions. Bands in the non-reducing condition correspond to intact Chi-44H10 or immunotargeting mAbs, and bands in the reducing condition correspond to the heavy and light chains of each mAb.

FIG. 5 shows flow cytometry data demonstrating the binding of chimeric 44H10 antibody conjugates to the lymphoblastoid B cell line BJAB. Chi-44H40 and immunotargeting mAbs were directly labeled using an Alexa 488 (A488) Maleimide dye and the binding was measured in the B530 channel. Gates on the histograms represent the positive signal established cells treated with the positive control (anti-CD19 antibody Denintuzumab).

FIG. 6A shows a structural model depicting the binding of three antibodies (CR3022, S309 and VHH-72) to three distinct conformational epitopes on the SARS-CoV-2 spike protein RBD. The Protein Data Bank (PDB) identification number corresponding to each antibody-RBD complex is specified next to each antibody.

FIG. 6B shows flow cytometry data demonstrating the binding of the three aforementioned antibodies to the RBD displayed on the immunotargeting mAbs. The immunotargeting mAbs were allowed to bind to BJAB cells, and then reacted with the anti-RBD antibodies fluorescently labeled using an Alexa 488 Maleimide dye. The binding was measured in the B530 channel. Gates on the histograms represent the positive signal established cells treated with the positive control (anti-CD19 antibody Denintuzumab).

FIG. 7A shows ELISA endpoint titer analysis depicting between immunization group comparisons of antibody titers at D49 elicited by soluble RBD (sRBD), Chi-44H10 and immunotargeting mAbs.

FIG. 7B shows ELISA data depicting the kinetics of anti-RBD antibody responses elicited by sRBD, Chi-44H10 and immunotargeting mAbs.

FIG. 8A shows SARS-CoV-2 Spike protein-expressing pseudovirus (wild-type) neutralization data comparing neutralization potency of serum antibodies elicited in rabbits immunized with sRBD, Chi-44H10 or immunotargeting mAbs at D49, D70 and D91. These data were fitted by non-linear regression.

FIG. 8B shows SARS-CoV-2 Spike protein-expressing pseudovirus neutralization data comparing neutralization potency of D49 serum antibodies elicited in rabbits immunized with immunotargeting mAbs against WIV04/2019 (wild-type), B.1.351 (beta), P.1 (gamma) and B.1.617.2 (delta) strains of SARS-CoV-2. These data were fitted by non-linear regression.

FIG. 9A shows ELISA data depicting the kinetics of anti-RBD antibody responses elicited by immunotargeting mAbs administered either subcutaneously (sub-Q) or intramuscularly (IM).

FIG. 9B shows SARS-CoV-2 Spike protein-expressing pseudovirus (wild-type) neutralization data comparing neutralization potency of serum antibodies elicited in rabbits immunized with immunotargeting mAbs administered either subcutaneously (sub-Q) or intramuscularly (IM) at D49, D70 and D91. These data were fitted by non-linear regression.

### DETAILED DESCRIPTION OF CERTAIN ASPECTS

In an effort to explore a possible extension of the utility of the immunotargeting vaccine strategy, we investigated the genetic incorporation of different universal T-helper determinants into the immunotargeting antigen complex, and unexpectedly found that they could provide significant benefit. Adding to the unexpected results was the observation that the extent of benefit from the incorporated T-helper determinant was dependent on the specific site of incorporation of the sequence in the immunotargeting antibody construct.

Previously there had been no indication that the immunotargeting approach to subunit vaccine design would benefit from the incorporation of a universal T-helper cell determinant, as these entities had been designed as synthetic peptides to be mixed with adjuvants. Because the immunotargeting approach to vaccine design is adjuvant-independent, it is counterintuitive to expect that these T-helper determinants, designed to work with adjuvants, might actually benefit the immunotargeting strategy.

Thus, described herein is the application and novel extension of the immunotargeting approach to vaccine design to create a vaccine for SARS-CoV-2. Specifically, the vaccine construct involves the receptor binding domain (RBD) of SARS-CoV-2 genetically fused to the C-terminus of the heavy chain of a chimeric humanized anti-class II MHC mAb, with a universal T-helper determinant genetically fused to the C-terminus of the light chain of the same mAb. When injected, this vaccine construct induces a potent, long-lived, virus-neutralizing IgG antibody response against the RBD of the SARS-CoV-2 Spike protein.

In aspects, these vaccine constructs utilize a recombinant monoclonal antibody (mAb) specific for a serological determinant widely expressed on human class II major histocompatibility complex (MHC) gene products. The receptor binding domain (RBD) of the SARS-CoV-2 virus Spike protein is genetically incorporated into the immunotargeting antibody to create the vaccine construct. In addition, specific sequences corresponding to universal T-helper determinants are also incorporated into the vaccine constructs. The results described herein indicate that rabbits immunized with these specific constructs induce potent and long-lived antibody responses which (using in vitro cellular infection assays) can be shown to neutralize virus expressing the corresponding SARS-CoV-2 Spike protein. They also indicate that certain anti-viral antibody responses are unexpectedly dependent on the specific sites of incorporation of the viral RBD and the T-helper sequences.

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in aspects, the compositions and vaccines described herein are free of an adjuvant or “adjuvant-free.”

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

A “vaccine” is a pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine induces an immune response that reduces the severity of the symptoms associated with SARS-CoV-2 infection and/or decreases the viral load compared to a control. In another non-limiting example, a vaccine induces an immune response that reduces and/or prevents SARS-CoV-2 infection compared to a control.

The term “antibody”, also referred to in the art as “immunoglobulin” (Ig), used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (VL) and a constant (CL) domain, while the heavy chain folds into a variable (VH) and three constant (C_{H}, C_{H2}, C_{H3}) domains. Interaction of the heavy and light chain variable domains (VH and VI) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of VL and VH connected with a peptide linker), Fab, F(ab′)₂, single domain antibody (sdAb; a fragment composed of a single V_{L} or V_{H}), and multivalent presentations of any of these.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “purified” means that impurities have been removed and the purified component is present at a higher concentration than it would otherwise be. For example, a composition comprising a purified component may comprise 85%, 90%, 95%, 96%, 97%, 98%, 99%, 99.5%, 99.9% or more of the component or 100% of the component in question.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

Furthermore, the term “broadly reactive” means that the antibody reacts or binds to a common (shared) genetic determinant or epitope expressed on multiple HLA-DR alleles in the human population. For example, the antibody may bind to any one or more of HLA-DR1, HLA-DR3, HLA-DR4, HLA-DR7, HLA-DR8, HLA-DR9, HLA-DR10, HLA-DR11, HLA-DR12, HLA-DR13, HLA-DR14, HLA-DR15, and/or HLA-DR16.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the antibodies described herein is, in aspects, sufficient to increase immunity against a pathogen, such as SARS-CoV-2.

Moreover, a treatment regime of a subject with a therapeutically effective amount may consist of a single administration, or alternatively comprise a series of applications. The length of the treatment period depends on a variety of factors, such as the immunogen, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The antibodies described herein may, in aspects, be administered before, during or after treatment with conventional therapies for the disease or disorder in question.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

The term “adjuvant” refers to a compound or mixture that is present in a vaccine and enhances the immune response to an antigen present in the vaccine. For example, an adjuvant may enhance the immune response to a polypeptide present in a vaccine as contemplated herein, or to an immunogenic fragment or variant thereof as contemplated herein. An adjuvant can serve as a tissue depot that slowly releases the antigen and also as a lymphoid system activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS021/AS02 (GSK). These immunostimulatory adjuvants are formulated to give a strong T cell response and include QS-21, a saponin from Quillay saponaria, the TL4 ligand, a monophosphoryl lipid A, together in a lipid or liposomal carrier. Other adjuvants include, but are not limited to, nonionic block co-polymer adjuvants (e.g., CRL 1005), aluminum phosphates (e.g., AIPO.sub.4), R-848 (a Th1-like adjuvant), imiquimod, PAM3CYS, poly (I:C), loxoribine, BCG (bacille Calmette-Guerin) and Corynebacterium parvum, CpG oligodeoxynucleotides (ODN), cholera toxin derived antigens (e.g., CTA 1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

“Variants” are biologically active proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the antibodies described herein, wherein “biological” activity refers to a biological function (either inhibitory or stimulatory) caused by the antibodies.

The proteins described herein may include modifications. Such modifications include, but are not limited to, conjugation to an effector molecule such as an anti-viral agent or an adjuvant. Modifications further include, but are not limited to conjugation to detectable reporter moieties. Modifications that extend half-life (e.g., pegylation) are also included. Proteins and non-protein agents may be conjugated to the antibodies by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

### Antibodies

In aspects, described herein is an anti-HLA-DR monoclonal antibody-based vaccine shown to induce strong, adjuvant-independent, virus-neutralizing IgG antibody responses to SARS-CoV-2. The observed anti-viral response is significantly enhanced in an unexpected way by adding a universal T-helper determinant in a specific location, the C-terminus of the antibody light chain. The observed enhancement, with each of two different T-helper determinant sequences, suggests that adding a T-helper determinant to an immunotargeting antibody construct in general could benefit the immune response observed for the targeted delivery of a variety of different antigens.

Thus, described herein in an anti-class II MHC antibody fused to a SARS-CoV-2 antigen. Typically, the is an anti-HLA-DR antibody. In typical aspects, the antibody is a broadly reactive anti-HLA-DR antibody. The antibody may be of any form or fragment but is typically an IgG, scFv, Fab′, Fab, F(ab′)₂, or scFab antibody. Typically, the antibody is an IgG antibody.

In typical aspects, the antibody is a monoclonal antibody, such as a 44H10 antibody, which specifically binds to a shared epitope on most or all HLA-DR molecules. Any other monoclonal antibody that has this same specificity could substitute for the 44H10 antibody.

The antibody may be of any species but is typically a chimeric mouse/human antibody, a humanized antibody, or a fully human antibody.

The SARS-CoV-2 antigen may be any antigen from the SARS-CoV-2 virus, such as a spike protein antigen or a nucleocapsid antigen. For example, the SARS-CoV-2 antigen may be an spike protein 51 antigen or an S2 antigen. Typically, the SARS-CoV-2 antigen is an RBD antigen.

It will be understood that the SARS-CoV-2 antigen may be fused to any part of the antibody, although typically it is fused away from the N-terminus to avoid inhibiting the antigen binding ability of the antibody. In typical aspects, the SARS-CoV-2 antigen is fused at or near the C-terminus of the heavy and/or light chain of the antibody. In some aspects, the SARS-CoV-2 antigen is fused to the heavy chain of the antibody and in some aspects, the SARS-CoV-2 antigen is fused to the light chain of the antibody. In typical aspects, wherein the antibody comprises two heavy chains and two light chains, a SARS-CoV-2 antigen is fused to each heavy chain or each light chain. In this case, the SARS-CoV-2 antigen may be independently the same or different but is typically the same. In other aspects, a SARS-CoV-2 antigen may be fused to one heavy chain and one light chain, two heavy chains, two light chains, all four heavy and light chains, or various combinations thereof. In other aspects, a plurality of SARS-CoV-2 antigens, being the same or different, may be fused to one or more antibody heavy or light chains, in series or parallel. Typically, the SARS-CoV-2 antigen is the RBD and typically an RBD is fused to each heavy chain at the C-terminus thereof. The RBD antigens may be the same or different and there may be 1, 2, 3, or 4 same or different antigens, such as RBD antigens.

Other moieties may be additionally fused to the antibody described herein. For example, the antibody may be fused to a universal T-helper determinant. Examples of a universal T-helper determinant include PADRE and/or TpD. Similar to the SARS-CoV-2 antigen, the universal T-helper determinant is typically fused at or near the C-terminus of the heavy chain and/or light chain of the antibody. In some aspects, the universal T-helper determinant is fused to the heavy chain of the antibody and in some aspects, the universal T-helper determinant is fused to the light chain of the antibody. In typical aspects, wherein the antibody comprises two heavy chains and two light chains, a universal T-helper determinant is fused to each heavy chain or each light chain. In this case, the universal T-helper determinant may be the same or different but is typically the same. In other aspects, a universal T-helper determinant may be fused to one heavy chain and one light chain, two heavy chains, two light chains, all four heavy and light chains, or various combinations thereof. In other aspects, a plurality of universal T-helper determinant antigens, being the same or different, may be fused to one or more antibody heavy or light chains, in series or parallel. Typically, the universal T-helper determinant is the PADRE sequence and typically a PADRE sequence is fused to each light chain at the C-terminus thereof. The universal T-helper determinants may be the same or different and there may be 1, 2, 3, or 4 same or different universal T-helper determinants, such as PADRE sequences.

It will be understood that linkers may be included that separate the antibody from another bound moiety, such as between the antibody and the SARS-CoV-2 antigen or the antibody and the universal T-helper determinant. Typically, the linker is a GS repeat linker, such as a GGS×2 linker (SEQ ID NO:16) or GGGGS×2 linker (SEQ ID NO:17).

In typical aspects, the antibody comprises a polypeptide sequence having at least 70% sequence identity to any one or more of the following:

1. Chimeric H chain + RBD:
(SEQ ID NO: 1)
MALLVLFLSLAAFPSCGVLSQVQLKESGPGLVAPSQSLSITCTVSGFSLTSYGVHWVRQPPGKGLE
WLGVIWAGGSINYNSALMSRLSISKDNFKSQVFLKMSSLQTDDTAMYYCARAYGDYVHYAMDYWGQ
GTSVTASSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLS
PGKGGGGSGGGGSRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNF*
Signal peptide
44H10 VH
Human CH (lgG1)
GGGGSx2 linker (SEQ ID NO: 17)
RBD (319-542)
2. Chimeric L chain + RBD:
(SEQ ID NO: 2)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGGGSGGGGSRVQPTESIVRFPNITNLCPFG
EVFNATRFASVYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPFERDISTEIYQA
GSTPCNGVEGFNCYFPLQSYGFQPTNGVGYQPYRVVVLSFELLHAPATVCGPKKSTNLVKNKCVNF*
Signal peptide
44H10 V_{K}
Human C_{K}
GGGGSx2 linker (SEQ ID NO: 17)
RBD (319-542)
3. Chimeric L chain + TpD:
(SEQ ID NO: 3)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGSGGSILMQYIKANSKFIGIPMGLPQSIALSSL
MVAQ*
Signal peptide
44H10 V_{K}
Human C_{K}
GGSx2 linker (SEQ ID NO: 16)
TpD
4. Chimeric L chain + PADRE:
(SEQ ID NO: 12)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGSGGSAKFVAAWTLKAAA*
Signal peptide
44H10 V_{K}
Human C_{K}
GGSx2 linker (SEQ ID NO: 16)
PADRE

These different sequences can be modular, in the sense that any of the portions in the above sequences can be swapped with other analogous sequences or omitted or additional sequences can be included. For example, a TpD sequence may be used instead of or in addition to a PADRE sequence or they may be swapped from heavy chain to light chain and different linkers can be used. Typically, the antibody comprises at least one heavy chain and at least one light chain of 1, 2, 3, and 4 listed above in any combination and, more typically, the antibody comprises or consists of two heavy and two light chains of 1, 2, 3, and 4 listed above, in any combination.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (IIe or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.orgitoolsiblast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The polypeptides or antibodies of the present invention may also comprise additional sequences to aid in their expression, detection or purification. Any such sequences or tags known to those of skill in the art may be used. For example, and without wishing to be limiting, the antibodies may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK (SEQ ID NO:18), Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or Hiss), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

The antibodies may also be in a multivalent display. Multimerization may be achieved by any suitable method of known in the art. For example, and without wishing to be limiting in any manner, multimerization may be achieved using self-assembly molecules as described in Zhang et al (2004a; 2004b) and WO2003/046560.

Also encompassed herein are isolated or purified antibodies, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the antibodies may be linked to a cargo molecule; the antibodies may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the antibody may be linked to a fluorescent agent such as FITC or may genetically be fused to the Enhanced Green Fluorescent Protein (EGFP).

The antibodies described herein specifically bind to their targets. Antibody specificity, which refers to selective recognition of an antibody fora particular epitope of an antigen, of the antibodies or fragments described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, or 10⁻⁹ M.

Also described herein are nucleic acid molecules encoding the antibodies and polypeptides described herein, as well as vectors comprising the nucleic acid molecules and host cells comprising the vectors.

Polynucleotides encoding the antibodies described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of DNAs that encode fragments of antibodies include any cell, such as hybridomas, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

The polynucleotides described herein may be used for example in vaccines, such as mRNA vaccines, as will be understood.

Additionally, the expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The antibodies described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce antibodies by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The antibodies described herein can be fused to additional amino acid residues. Such amino acid residues can be a peptide tag to facilitate isolation, for example. Other amino acid residues for homing of the antibodies to specific organs or tissues are also contemplated.

In another aspect, described herein are methods of vaccinating subjects by administering a therapeutically effective amount of the antibodies or vaccines described herein to a mammal in need thereof, typically an adult, elderly, young, juvenile, or neonatal mammal. Therapeutically effective means an amount effective to produce the desired therapeutic effect, such as providing a protective immune response against the antigen in question.

Any suitable method or route can be used to administer the antibodies and vaccines described herein. Routes of administration include, for example, oral, intravenous, intraperitoneal, subcutaneous, or intramuscular administration.

It is understood that the antibodies described herein, where used in a mammal for the purpose of prophylaxis or treatment, will be typically administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be administered to other mammals as well. The term “mammal” as used herein is intended to include, but is not limited to, humans, laboratory animals, domestic pets and farm animals.

Also included herein are kits for vaccination, comprising a therapeutically or prophylactically effective amount of the antibodies described herein. The kits can further contain any suitable adjuvant for example or, in aspects, they exclude an adjuvant. Kits may include instructions.

Also described are methods of immunizing a subject against SARS-CoV-2 as well as methods of treating and/or preventing SARS-CoV-2 in a subject. Also described are related uses of the SARS-CoV-2 antibody or vaccine described herein for immunizing a subject against SARS-CoV-2 and/or for treating and/or preventing SARS-CoV-2.

These methods and uses comprise administering the SARS-CoV-2 antibody or vaccine to a subject infected with SARS-CoV-2, a subject suspected of being infected with SARS-CoV-2, or a subject at risk of being infected with SARS-CoV-2.

In aspects, the methods and uses further comprise administering a vaccine against tetanus and/or diphtheria toxoids to the subject and/or are carried out in a subject previously vaccinated against tetanus and/or diphtheria toxoids. Many examples of such vaccinations against tetanus and/or diphtheria toxoids exist, such as the Td Adsorbed vaccine available from Sanofi Pasteur. For example, if a potential vaccine recipient were immunized with Td one month prior to receiving a immunotargeted SARS-CoV-2 vaccine which had TpD incorporated into the construct, the T-helper cells induced in the individual by the Td vaccine could act to significantly enhance the antibody response to the SARS-CoV-2 antigens on the vaccine. This could provide an enhancement in terms of anti-SARS-CoV-2 antibody responses in certain populations, such as the elderly, the immunocompromised, or other populations that may otherwise be poor responders.

In aspects, the vaccine against tetanus and/or diphtheria toxoids may administered to the subject substantially simultaneously with or prior to the SARS-CoV-2 vaccine, such as one or more days, weeks, months, or years prior to the SARS-CoV-2 vaccine, such as about one month prior to the SARS-CoV-2 vaccine.

While the SARS-CoV-2 vaccine may be use together with an adjuvant, it will be understood that, in typical aspects, the SARS-CoV-2 vaccine is administered without an adjuvant. In typical aspects, the SARS-CoV-2 vaccine is administered as a purified protein without an adjuvant.

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1: This Example Illustrates the Co-Transfection of Plasmids Encoding Antibody Heavy and Light Chains into Freestyle 293-F Cells for the Expression of Immunotargeting mAbs

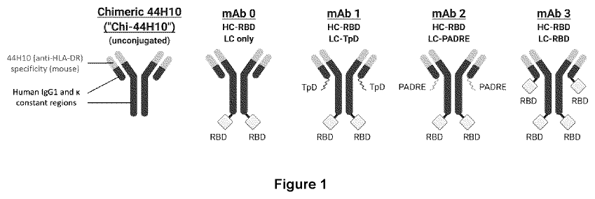

FIG. 1 shows schematics of the unconjugated chimeric human IgG1 anti-HLA-DR 44H10 (mouse VH and VL) antibody (“Chi-44H10”) and four different immunotargeting mAbs with SARS-CoV-2 Spike protein Receptor Binding Domain (RBD) and universal T-helper determinants (TpD or PADRE) integrated into either immunoglobulin heavy (HC) or light (LC) chains as indicated (44H10 as described by Dubiski et al. (1988)¹⁰).

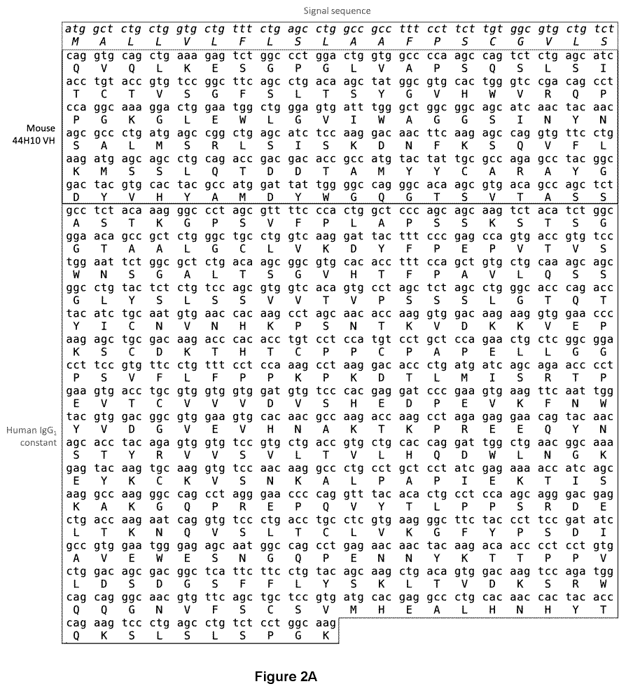

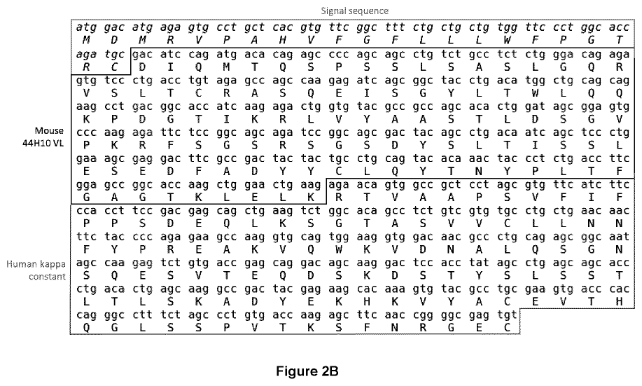

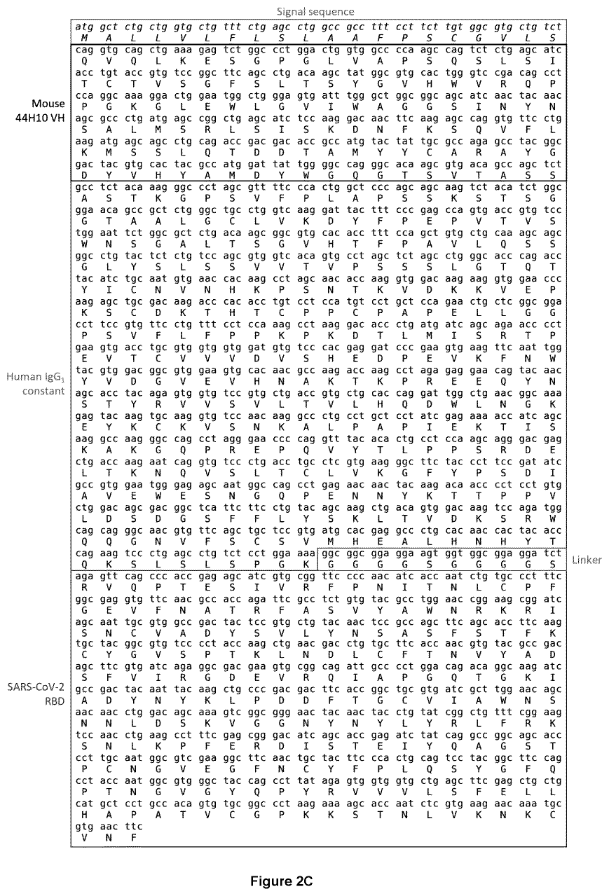

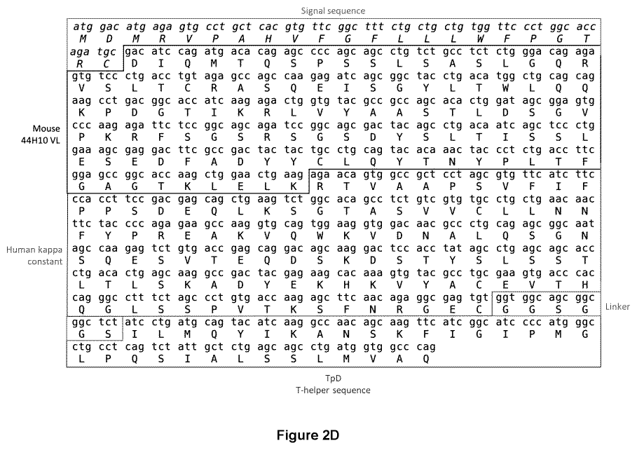

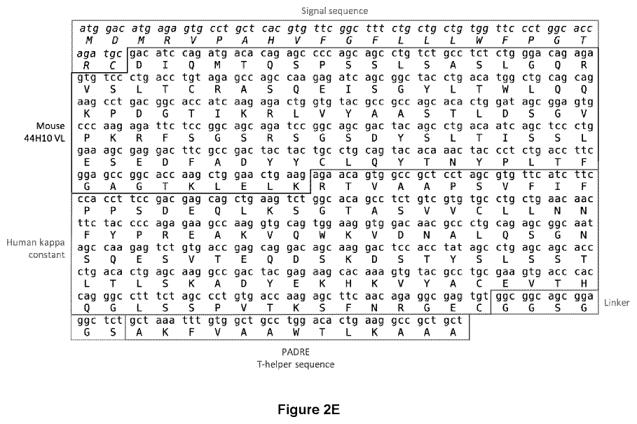

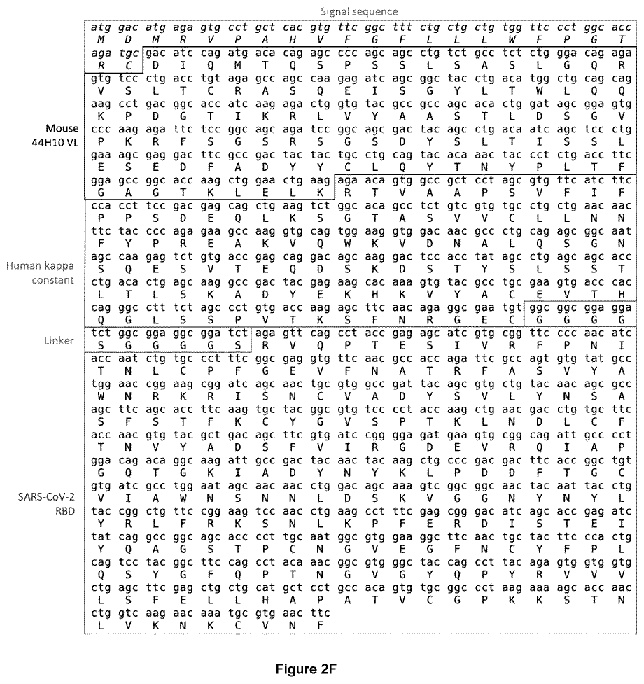

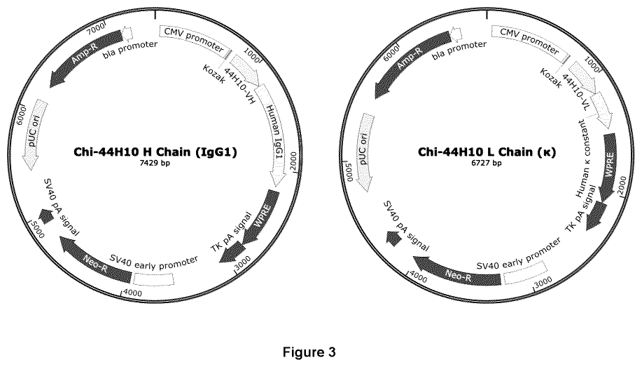

DNA plasmids encoding heavy and light chain antibody constructs were designed in the pcDNA3.4 TOPO expression vector and optimized for Homo sapiens expression using GeneArt Gene Synthesis (Invitrogen) (FIGS. 2 and 3). These constructs were maxiprepped using PureLink HiPure Plasmid Maxiprep Kits (Invitrogen).

FreeStyle 293-F Cells were cultured in 500 mL polycarbonate Erlenmeyer flasks (Triforest Labware) in FreeStyle 293 Expression Medium (Gibco) and split to a density of 0.8×10⁶ cells/ml at least one hour before transfection. Cells were transfected using the FectoPRO Reagent (Polyplus) following manufacturer instructions at a 1:1 DNA to FectoPRO ratio. 90 ug of plasmid DNA was used for transfection (60 ug of heavy chain DNA and 30 ug of light chain DNA) for every 200 mL of cell culture. Transfected cells were incubated in a 37° C., 5% CO2 shaking incubator for 5 to 7 days to allow for the expression and self-assembly of heavy and light chain gene products.

### Example 2. This Example Illustrates the Purification of Immunotargeting mAbs from the Supernatant of Transfected FreeStyle 293-F Cells

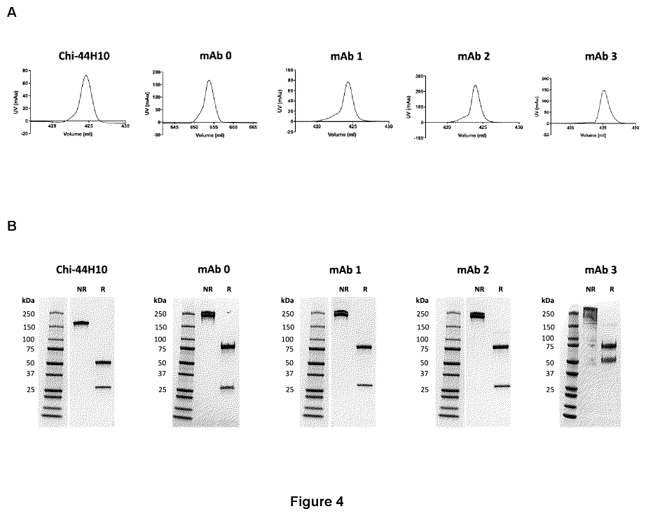

Transfected cell culture supernatants were collected and filtered through 0.22 μM Steritop filters (Millipore Sigma) before loading onto protein A affinity columns using the ÄKTA start protein purification system (Cytiva Life Sciences). Following loading, samples were washed with 1X phosphate-buffered saline (PBS) then eluted with 100 mM glycine pH 2.2 and immediately neutralized with 1 M Tris pH 9. Samples collected from the elution peaks (FIG. 4A) were buffer exchanged into PBS using PD-10 desalting columns (Cytiva Life Sciences) and adjusted to a final concentration of 1 mg/ml using Nanodrop 2000 Spectrophotometer measurements (Thermo Scientific).

Samples were run on 10-well 4-20% SDS-PAGE gradient gels in non-reducing and reducing sample buffer (±2-Mercaptoethanol) at 250 V for 20 minutes (Bio-Rad). Gels were stained with Coomassie Brilliant Blue for protein visualization (FIG. 4B). A band corresponding to the intact Chi-44H10 antibody is detected in the non-reducing condition at 150 kDa, and individual heavy and light chain bands in the reducing condition are detected at 50 kDa and 25 kDa, respectively. Bands for the immunotargeting mAbs bearing heavy and/or light chain conjugates are shifted to increased molecular weights relative to the unmodified Chi-44H10 bands in accordance with the size of the added entity.

### Example 3. This Example Illustrates the Procedure for the Fluorescent Labeling of Unconjugated and Conjugated Antibodies for Subsequent Flow Cytometric Experiments

Purified antibodies were diluted at a concentration of 100 μM in PBS and incubated in a 10-fold molar excess of TCEP at room temperature for 30 minutes to reduce disulfide bonds and render cysteines accessible for labeling. After 30 minutes, Alexa Fluor C₅ Maleimide dye (Invitrogen) was added to each reaction at a concentration of 10 mM for a 10-20-molar excess of dye to protein. Samples were incubated overnight at 4° C. protected from light. Free, unconjugated dye was washed out of solution using PBS and Amicon 30K Ultra-0.5 mL Centrifugal Filters (Millipore Sigma) and the concentration of labeled protein was assessed by Nanodrop measurement.

### Example 4. This Example Illustrates the Flow Cytometric Procedure for the Assessment of Fluorescently Labeled Immunotargeting mAb Binding to the B-Lymphoblastoid Cell Line BJAB

B lymphoblastoid BJAB cells (Thermo Scientific) (described in Menezes et al. (1975)¹¹) were grown in supplemented RPMI medium containing 10% fetal bovine serum (FBS) in a 37° C., 5% CO2 incubator. Cells were collected in a 15 ml conical tube and centrifuged at 300 g for 5 minutes. Cell pellets were resuspended in staining buffer (PBS containing 2% FBS and 0.05% NaN₃) at a concentration of 1×10⁶ cells/ml, and 200 μl of the cell suspension was dispensed into the wells of a polystyrene, V-bottom 96-well plate (Greiner Bio-One) for staining. The plate was centrifuged at 300 g for 5 minutes, and the cells were resuspended in an Fc-block solution and incubated at 4° C. for 30 minutes. Cells were washed once in staining buffer to remove the Fc block solution. The cells were resuspended in 50 ul of 0.1 ug/ul pre-labeled (A488) antibody (per described in Example 3) and incubated at 4° C. for 1 hour. The cells were washed twice in staining buffer, then resuspended in staining buffer containing 0.5 μM DAPI (PromoCell) for the exclusion of dead cells and debris. A control consisting of cells incubated with a labeled, unrelated isotype-matched antibody served as a negative control in this experiment.

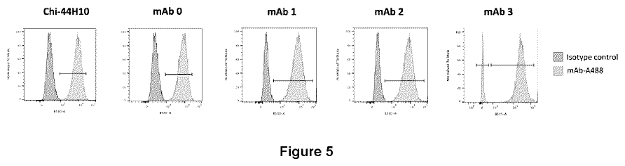

The binding of fluorescent anti-HLA-DR antibodies to BJAB cells was analyzed in a BD LSR II cytometer in the B530 channel. Gates on the histograms represent the positive signal established by the positive control (anti-CD19 antibody Denintuzumab). The binding of fluorescently labeled Chi-44H10 and immunotargeting mAbs to BJAB cells is depicted in FIG. 5, validating the ability of these antibodies to target HLA-DR expressed on B-lymphoblastoid cells.

### Example 5. This Example Illustrates the Flow Cytometric Procedure for the Assessment of RBD Structural Integrity on Immunotargeting mAbs Using Fluorescently Labeled Anti-RBD Antibodies

The structural integrity of the SARS-CoV-2 RBD attachment on the immunotargeting mAbs was assessed by the same technique described in Example 4, using three antibodies targeting distinct sites of the RBD as conformational probes: CR3022^{12,13}, S309¹⁴ and VHH72¹⁵. For this analysis, BJAB cells were first incubated with 50 ul of 0.1 ug/ul unlabeled purified Chi-44H10 or immunotargeting mAbs, washed once, then stained with 50 ul of 0.1 ug/ul pre-labeled (A488) CR3022, S309 and VHH72-Fc antibodies. A set of cells only incubated with labelled anti-RBD antibodies in the absence of immunotargeting mAbs served as a negative control.

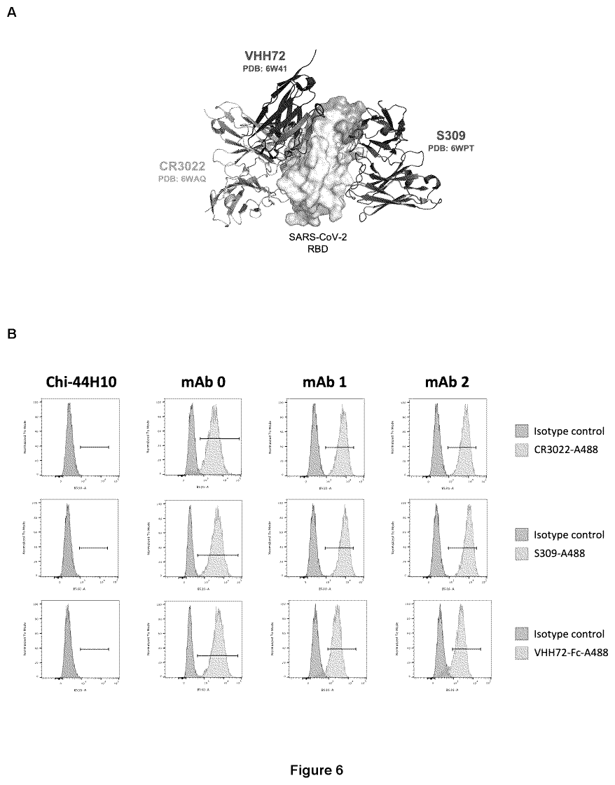

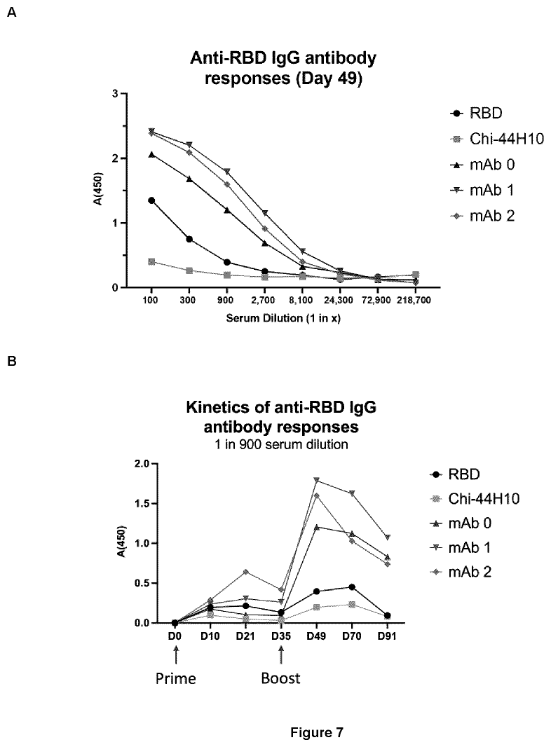

The binding of fluorescently labeled anti-RBD antibodies to immunotargeting mAbs bound to BJAB cells, but not direct binding to BJAB cells, is depicted in FIG. 6. This indicates that RBD epitopes on the immunotargeting mAbs are structurally intact and accessible. No anti-RBD antibody binding to unmodified Chi-44H10 was detected, as this antibody lacks any RBD.

### Example 6. This Example Illustrates the Procedure for the Immunization of Animals with Immunotargeting mAbs

Immunogens were diluted to a concentration of 0.05 mg/ml in PBS. Female New Zealand white rabbits (5 per group) were immunized by Cedarlane Laboratories with 50 ug of unadjuvanted immunogen via subcutaneous or intramuscular injection, followed by a boost of the same dose at 5 weeks post-prime (D35). A control group immunized with soluble RBD was used to examine the specific effect of immunotargeting by anti-HLA-DR antibody conjugation, using a dose corresponding to an equimolar amount of RBD compared to the immunotargeting mAbs (i.e. 7.5 μg).

Rabbits were bled before immunization (DO) and at days 10, 21, 35, 49, 70 and 92 post-primary immunization (Table 1).

TABLE 1
(immediately below) outlines the dosage and schedule set forth
for rabbit immunizations with soluble RBD (sRBD), Chi-44H10 or
immunotargeting mAbs. For subcutaneous administration, animals
were injected with ¼ of the total dose at 4 separate
sites for both the prime and boost immunizations. For intramuscular
administration, animals were injected with the total dose at a
single site for both the prime and boost immunizations. Note that
the selected dose of 7.5 ug of sRBD is equimolar to the amount
of RBD in 50 ug of each immunotargeting antibody.

| | |
|---|---|
| Day 0 | Pre-immune bleed |
| | Primary immunization (50 ug of antibody or 7.5 ug of RBD) |
| Day 10 | Bleed 1 |
| Day 21 | Bleed 2 |
| Day 35 | Bleed 3 |
| | Booster immunization (50 ug of antibody or 7.5 ug of RBD) |
| Day 49 | Bleed 4 |
| Day 70 | Bleed 5 |
| Day 91 | Bleed 6 |

### Example 7. This Example Illustrates the Procedure for the Collection of Blood from Rabbits and Preparation of Serum for Subsequent Assays

The following procedures were performed at Cedarlane Laboratories in Burlington, ON, Canada. Blood was collected into red top vacutainer tubes and incubated at 3-4 hours at room temperature to allow for clotting. The tubes were centrifuged at 4° C. at ˜3000 rpm for 20 minutes to pellet the red blood cells and debris. Supernatants were poured off into appropriate tubes and stored at −20° C. before shipping on dry ice.

### Example 8. This Example Illustrates the ELISA Procedure for the Assessment of Serum Anti-SARS-CoV-2 RBD Elicited by Immunization with Immunotargeting mAbs

Immulon 4 HBX ELISA plates (Thermo Scientific) were coated with 50 μl of 2 ug/mL SARS-CoV-2 RBD diluted in PBS and incubated overnight at 4° C. The coating solution was removed and plates were washed three times with PBS-T (PBS containing 0.1% Tween). Plates were incubated with blocking buffer (PBS-T containing 3% non-fat milk) for 1 hour at room temperature. Serum samples were serially pre-diluted in diluent buffer (PBS-T containing 1% non-fat milk) in 1.2 mL microtiter dilution tubes (Thermo Fisher). The blocking solution was discarded and 100 μl of pre-diluted sera was added to the plates and incubated at room temperature for 2 hours. Plates were washed three times with PBS-T and incubated with Goat Anti-Rabbit IgG H&L (HRP) secondary antibody (Abcam 97051) at a 1:10,000 dilution for 1 hour at room temperature. Plates were developed using a TMB Substrate Reagent Set (BD) following manufacturer instructions; reactions were stopped at 10 minutes by the addition of 2N HCl. Plates were read at an absorbance of 450 nm using a Synergy Neo2 Multi-Mode Assay Microplate Reader (Biotek Instruments).

### Example 9. This Example Illustrates the Procedure for the Pseudovirus Neutralization Assay Used to Assess Neutralization Potency of Antibodies Elicited by Immunization with Immunotargeting mAbs

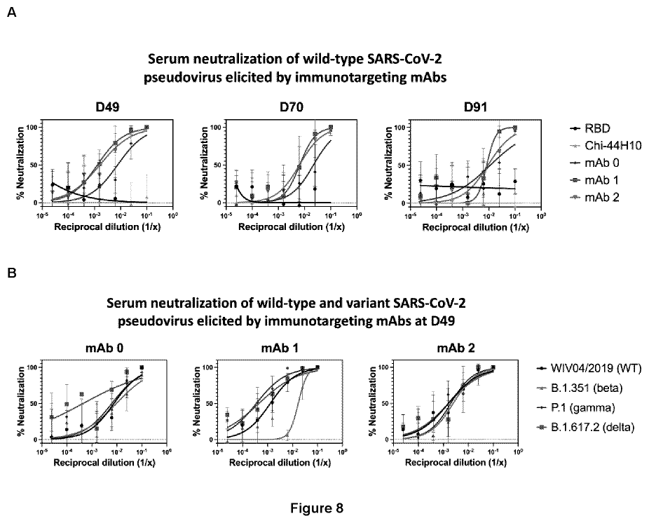

The procedure described in this Example was adapted from Crawford et al. (2020)¹⁶. 293T cells were co-transfected with a lentiviral backbone encoding the luciferase reporter gene (BEI NR52516), a plasmid expressing the SARS-CoV-2 Spike (BEI NR52310) and plasmids encoding the HIV structural and regulatory proteins Tat (BEI NR52518), Gag-pol (BEI NR52517) and Rev (BEI NR52519). Co-transfection of the five plasmids was performed using BioT reagent (Bioland Scientifics) following manufacturer instructions. 24 hours post-transfection at 37° C., the media was supplemented with 5 mM sodium butyrate (NaB) and the cells were further incubated for an additional 24 hours at 30° C. prior to pseudovirus (PsV) harvesting. The neutralization assay was performed using 293T-ACE2 cells (BEI NR52511) as previously described¹⁶ with few modifications. Briefly, rabbit sera was inactivated by 30-minute incubation at 56° C. 4-fold serial dilutions of the inactivated sera were incubated for 1 hour at 37° C. with SARS-CoV-2 PsV and subsequently added to 293T-ACE2 cells (BEI NR52511) seeded in Poly-L-lysine (Sigma-Aldrich) coated plates 24 hours prior to the experiment. After 48 hours of incubation, PsV neutralization was monitored adding 50 μl of Britelite plus reagent (PerkinElmer) to 50 μl of cells. After 2 minutes of incubation, the volume was transferred to a 96-well white plate (Sigma-Aldrich) to measure luminescence in relative light units (RLUs) using a Synergy Neo2 Multi-Mode Assay Microplate Reader (Biotek Instruments). The data were analyzed by non-linear regression (FIG. 8, Tables 2 and 3). It has now been established that the best correlate of Covid-19 vaccine efficacy in humans is the presence of virus-neutralizing antibodies in the serum of vaccine recipients (17). Therefore the virus-neutralizing antibody responses described in Example 9 (FIGS. 8 & 9, Tables 2, 3 & 4) represent a clear indication of the vaccine properties of the modified antibody molecules for SARS-CoV-2.

TABLE 2
(immediately below) outlines the half maximal Inhibitory Concentration
(IC50) values for the pseudovirus neutralization data shown
in FIG. 8A, indicating the serum dilution at which 50% of wild-
type pseudovirus is neutralized in the assay. Note that no pseudovirus
neutralization was detected in the pre-boost (D0, D10, D21
and D35) serum of any immunization group.

| | D49 | D70 | D91 |
|---|---|---|---|
| sRBD | — | — | — |
| Chi-44H10 | — | — | — |
| mAb 0 | 1:130 | 1:45 | 1:88 |
| mAb 1 | 1:778 | 1:153 | 1:120 |
| mAb 2 | 1:627 | 1:157 | 1:108 |

TABLE 3
(immediately below) outlines the half maximal Inhibitory
Concentration (IC50) values for the pseudovirus neutralization
data shown in FIG. 8B, indicating the serum dilution
at which 50% of pseudovirus of the specified strain is
neutralized at D49 post-primary immunization.

| | WIV04/2019 | B.1.351 | P.1 | B.1.617.2 |
|---|---|---|---|---|
| | Wild-type | Beta variant | Gamma variant | Delta variant |
| mAb 0 | 1:130 | 1:103 | 1:158 | 1:2227 |
| mAb 1 | 1:778 | 1:59 | 1:2807 | 1:2212 |
| mAb 2 | 1:627 | 1:401 | 1:501 | 1:617 |

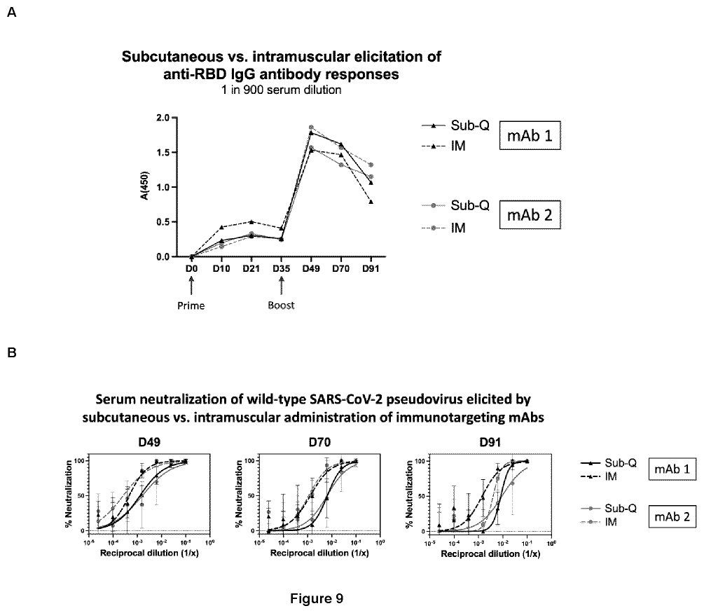

TABLE 4
(immediately below) outlines the half maximal Inhibitory Concentration
(IC50) values for the pseudovirus neutralization data shown in
FIG. 9B comparing subcutaneous (sub-Q) and intramuscular (IM)
administration routes, indicating the serum dilution at which
50% of wild-type pseudovirus is neutralized in the assay. Note
that no pseudovirus neutralization was detected in the pre-boost
(D0, D10, D21 and D35) serum of any immunization group.

| | | D49 | D70 | D91 |
|---|---|---|---|---|
| mAb 1 | Sub-Q | 1:778 | 1:153 | 1:120 |
| | IM | 1:2173 | 1:778 | 1:706 |
| mAb 2 | Sub-Q | 1:627 | 1:157 | 1:109 |
| | IM | 1:3832 | 1:905 | 1:249 |

### REFERENCES

1. Janeway, C. A. How the immune system protects the host from infection. Microbes and Infection vol. 3 1167-1171 (2001).
2. Petrovsky, N. Comparative Safety of Vaccine Adjuvants: A Summary of Current Evidence and Future Needs. Drug Safety vol. 38 1059-1074 (2015).
3. Carayanniotis, G. & Barber, B. H. Adjuvant-free IgG responses induced with antigen coupled to antibodies against class II MHC. Nature 327, 59-61 (1987).
4. Barber, B. H. The immunotargeting approach to adjuvant-independent subunit vaccine design. Semin. Immunol. 9, 293-301 (1997).
5. Keler, T., He, L., Ramakrishna, V. & Champion, B. Antibody-targeted vaccines. Oncogene 26, 3758-3767 (2007).
6. Gil, F. et al. Targeting antigens to an invariant epitope of the MHC Class II DR molecule potentiates the immune response to subunit vaccines. Virus Res. 155, 55-60 (2011).
7. Knutson, K. L. & Disis, M. L. Tumor antigen-specific T helper cells in cancer immunity and immunotherapy. Cancer Immunology, Immunotherapy vol. 54 721-728 (2005).
8. Alexander, J. et al. Development of high potency universal DR-restricted helper epitopes by modification of high affinity DR-blocking peptides. Immunity 1, 751-761 (1994).
9. Fraser, C. C. et al. Generation of a universal CD4 memory T cell recall peptide effective in humans, mice and non-human primates. Vaccine 32, 2896-2903 (2014).
10. Dubiski, S., Cinader, B., chou, C. T., Charpentier, L. & Letarte, M. Cross-reaction of a monoclonal antibody to human MHC class II molecules with rabbit B cells. Mol. Immunol. 25, 713-718 (1988).
11. Menezes, J., Leibold, W., Klein, G. & Clements, G. Establishment and characterization of an Epstein-Barr virus (EBC)—negative lymphoblastoid B cell line (BJA-B) from an exceptional, EBV-genome-negative African Burkitt's lymphoma. Biomedicine 22, 276-284 (1975).
12. Yuan, M. et al. A highly conserved cryptic epitope in the receptor binding domains of SARS-CoV-2 and SARS-CoV. Science (80-.). 368, 630-633 (2020).
13. Tian, X. et al. Potent binding of 2019 novel coronavirus spike protein by a SARS coronavirus-specific human monoclonal antibody. Emerging Microbes and Infections vol. 9 382-385 (2020).
14. Pinto, D. et al. Cross-neutralization of SARS-CoV-2 by a human monoclonal SARS-CoV antibody. Nature (2020) doi:10.1038/s41586-020-2349-y.
15. Wrapp, D. et al. Structural Basis for Potent Neutralization of Betacoronaviruses by Single-Domain Camelid Antibodies. Cell 181, 1004-1015.e15 (2020).
16. Crawford, K. H. et al. Protocol and reagents for pseudotyping lentiviral particles with SARS-CoV-2 Spike protein for neutralization assays. bioRxiv 2020.04.20.051219 (2020) doi:10.1101/2020.04.20.051219.
17. Earle, K. A. et al. Evidence for antibody as a protective correlate for COVID-19 vaccines. Vaccine 39, 4423 (2021).

1. An anti-class II MHC antibody fused to a SARS-CoV-2 antigen.

2. The antibody of claim 1, wherein the antibody is an anti-HLA-DR antibody.

3. The antibody of claim 2, wherein the antibody is a broadly reactive anti-HLA-DR antibody.

4. The antibody of claim 1, wherein the antibody is an IgG, scFv, Fab′, Fab, F(ab′)₂, or scFab.

5. (canceled)

6. The antibody of claim 1, wherein the antibody is a monoclonal antibody.

7. The antibody of claim 1, wherein the antibody has a 44H10 specificity.

8-9. (canceled)

10. The antibody of claim 1, wherein the antibody is humanized or human.

11-12. (canceled)

13. The antibody of claim 1, wherein the SARS-CoV-2 antigen is an RBD antigen.

14. The antibody of claim 1, wherein the SARS-CoV-2 antigen is fused to a heavy chain or a light chain of the antibody.

15. The antibody of claim 14, wherein the SARS-CoV-2 antigen is fused at the C-terminus of the heavy chain or the light chain.

16-17. (canceled)

18. The antibody of claim 1, wherein the antibody comprises two heavy chains and/or two light chains and a plurality of SARS-CoV-2 antigens, either the same or different, each fused to a different heavy chain and/or light chain.

19-24. (canceled)

25. The antibody of claim 1, further comprising a universal T-helper determinant.

26. The antibody of claim 25, wherein the universal T-helper determinant comprises PADRE and/or TpD.

27-30. (canceled)

31. The antibody of claim 1, wherein the antibody comprises two heavy chains and/or two light chains and comprising a plurality of universal T-helper determinants, either the same or different, each fused to a different heavy chain and/or light chain.

32-36. (canceled)

37. The antibody of claim 1 comprising a polypeptide sequence having at least 70% sequence identity to any one or more of:
1. Chimeric H chain + RBD:
(SEQ ID NO: 1)
MALLVLFLSLAAFPSCGVLSQVQLKESGPGLVAPSQSLSITCTVSGFSLTSYGVHWVRQPPGKGLE
WLGVIWAGGSINYNSALMSRLSISKDNFKSQVFLKMSSLQTDDTAMYYCARAYGDYVHYAMDYWGQ
GTSVTASSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLS
PGKGGGGSGGGGSRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNF*
Signal peptide
44H10 VH
Human CH (lgG1)
GGGGSx2 linker (SEQ ID NO: 17)
RBD (319-542)
2. Chimeric L chain + RBD:
(SEQ ID NO: 2)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGGGSGGGGSRVQPTESIVRFPNITNLCPFG
EVFNATRFASVYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPFERDISTEIYQA
GSTPCNGVEGFNCYFPLQSYGFQPTNGVGYQPYRVVVLSFELLHAPATVCGPKKSTNLVKNKCVNF*
Signal peptide
44H10 V_{K}
Human C_{K}
GGGGSx2 linker (SEQ ID NO: 17)
RBD (319-542)
3. Chimeric L chain + TpD:
(SEQ ID NO: 3)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGSGGSILMQYIKANSKFIGIPMGLPQSIALSSL
MVAQ*
Signal peptide
44H10 V_{K}
Human C_{K}
GGSx2 linker (SEQ ID NO: 16)
TpD
4. Chimeric L chain + PADRE:
(SEQ ID NO: 12)
MDMRVPAHVFGFLLLWFPGTRCDIQMTQSPSSLSASLGQRVSLTCRASQEISGYLTWLQQKPDGTI
KRLVYAASTLDSGVPKRFSGSRSGSDYSLTISSLESEDFADYYCLQYTNYPLTFGAGTKLELKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGSGGSAKFVAAWTLKAAA*
Signal peptide
44H10 V_{K}
Human C_{K}
GGSx2 linker (SEQ ID NO: 16)
PADRE

38. The antibody of claim 37, comprising;
a) at least one heavy chain and at least one light chain of 1, 2, 3, and 4, in any combination;
b) comprising two heavy chains and two light chains of 1, 2, 3, and 4, in any combination; or
c) consisting of two heavy chains and two light chains of 1, 2, 3, and 4, in any combination.

39-40. (canceled)

41. A polynucleotide encoding the antibody of claim 1.

42. A vaccine comprising the polynucleotide of claim 41.

43-44. (canceled)

45. A SARS-CoV-2 vaccine comprising the antibody of claim 1.

46. The SARS-CoV-2 vaccine of claim 45, wherein the SARS-CoV-2 vaccine is free of an adjuvant.

47. (canceled)

48. A method of treating and/or preventing SARS-CoV-2 in a subject, the method comprising administering the SARS-CoV-2 vaccine of claim 42 to the subject.

49. The method of claim 47, further comprising administering a vaccine against tetanus and/or diphtheria toxoids to the subject.

50-51. (canceled)

52. The method of claim 47, wherein the SARS-CoV-2 vaccine is administered without an adjuvant.

53-59. (canceled)


SEQUENCE LISTING (18 sequences)

SEQ ID NO: 1 - protein, 703 aa, Artificial Sequence
  Chimeric H chain + RBD
MALLVLFLSLAAFPSCGVLSQVQLKESGPGLVAPSQSLSITCTVSGFSLTSYGVHWVRQPPGKGLEWLGVIWAGGSINYNSALMSRLSISKDNFKSQVFLKMSSLQTDDTAMYYCARAYGDYVHYAMDYWGQGTSVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 469 aa, Artificial Sequence
  Chimeric L chain + RBD
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 274 aa, Artificial Sequence
  Chimeric L chain + TpD
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSILMQYIKANSKFIGIPMGLPQSIALSSLMVAQ

SEQ ID NO: 4 - protein, 470 aa, Artificial Sequence
  Chimeric H chain
MALLVLFLSLAAFPSCGVLSQVQLKESGPGLVAPSQSLSITCTVSGFSLTSYGVHWVRQPPGKGLEWLGVIWAGGSINYNSALMSRLSISKDNFKSQVFLKMSSLQTDDTAMYYCARAYGDYVHYAMDYWGQGTSVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1410 bp, Artificial Sequence
  Chimeric H chain
atggctctgctggtgctgtttctgagcctggccgcctttccttcttgtggcgtgctgtctcaggtgcagctgaaagagtctggccctggactggtggccccaagccagtctctgagcatcacctgtaccgtgtccggcttcagcctgacaagctatggcgtgcactgggtccgacagcctccaggcaaaggactggaatggctgggagtgatttgggctggcggcagcatcaactacaacagcgccctgatgagccggctgagcatctccaaggacaacttcaagagccaggtgttcctgaagatgagcagcctgcagaccgacgacaccgccatgtactattgcgccagagcctacggcgactacgtgcactacgccatggattattggggccagggcacaagcgtgacagccagctctgcctctacaaagggccctagcgttttcccactggctcccagcagcaagtctacatctggcggaacagccgctctgggctgcctggtcaaggattactttcccgagccagtgaccgtgtcctggaattctggcgctctgacaagcggcgtgcacacctttccagctgtgctgcaaagcagcggcctgtactctctgtccagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgctccgtgatgcacgaggccctgcacaaccactacacccagaagtccctgagcctgtctcctggcaag

SEQ ID NO: 6 - protein, 236 aa, Artificial Sequence
  Chimeric L chain
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 708 bp, Artificial Sequence
  Chimeric L chain
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaaccggggcgagtgt

SEQ ID NO: 8 - protein, 703 aa, Artificial Sequence; identical to SEQ ID NO: 1
  Chimeric H chain + RBD

SEQ ID NO: 9 - DNA, 2109 bp, Artificial Sequence
  Chimeric H chain + RBD
atggctctgctggtgctgtttctgagcctggccgcctttccttcttgtggcgtgctgtctcaggtgcagctgaaagagtctggccctggactggtggccccaagccagtctctgagcatcacctgtaccgtgtccggcttcagcctgacaagctatggcgtgcactgggtccgacagcctccaggcaaaggactggaatggctgggagtgatttgggctggcggcagcatcaactacaacagcgccctgatgagccggctgagcatctccaaggacaacttcaagagccaggtgttcctgaagatgagcagcctgcagaccgacgacaccgccatgtactattgcgccagagcctacggcgactacgtgcactacgccatggattattggggccagggcacaagcgtgacagccagctctgcctctacaaagggccctagcgttttcccactggctcccagcagcaagtctacatctggcggaacagccgctctgggctgcctggtcaaggattactttcccgagccagtgaccgtgtcctggaattctggcgctctgacaagcggcgtgcacacctttccagctgtgctgcaaagcagcggcctgtactctctgtccagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgctccgtgatgcacgaggccctgcacaaccactacacccagaagtccctgagcctgtctcctggaaaaggcggcggaggaagtggtggcggaggatctagagttcagcccaccgagagcatcgtgcggttccccaacatcaccaatctgtgccctttcggcgaggtgttcaacgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgactacaattacaagctgcccgacgacttcaccggctgcgtgatcgcttggaacagcaacaacctggacagcaaagtcggcgggaactacaactacctgtatcggctgtttcggaagtccaacctgaagcctttcgagcgggacatcagcaccgagatctatcaggccggcagcaccccttgcaatggcgtcgaaggcttcaactgctacttcccactgcagtcctacggcttccagcctaccaatggcgtgggctaccagccttatagagtggtggtgctgagcttcgagctgctgcatgctcctgccacagtgtgcggccctaagaaaagcaccaatctcgtgaagaacaaatgcgtgaacttc

SEQ ID NO: 10 - protein, 274 aa, Artificial Sequence; identical to SEQ ID NO: 3
  Chimeric L chain + TpD

SEQ ID NO: 11 - DNA, 822 bp, Artificial Sequence
  Chimeric L chain + TpD
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaacagaggcgagtgtggtggcagcggcggctctatcctgatgcagtacatcaaggccaacagcaagttcatcggcatccccatgggcctgcctcagtctattgctctgagcagcctgatggtggcccag

SEQ ID NO: 12 - protein, 255 aa, Artificial Sequence
  Chimeric L chain + PADRE
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAA

SEQ ID NO: 13 - DNA, 765 bp, Artificial Sequence
  Chimeric L chain + PADRE
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaacagaggcgagtgtggcggcagcggaggctctgctaaatttgtggctgcctggacactgaaggccgctgct

SEQ ID NO: 14 - protein, 469 aa, Artificial Sequence; identical to SEQ ID NO: 2
  Chimeric L chain + RBD

SEQ ID NO: 15 - DNA, 1407 bp, Artificial Sequence
  Chimeric L chain + RBD
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaacagaggcgaatgtggcggcggaggatctggcggaggcggatctagagttcagcctaccgagagcatcgtgcggttccccaacatcaccaatctgtgccctttcggcgaggtgttcaacgccaccagattcgccagtgtgtatgcctggaaccggaagcggatcagcaactgcgtggccgattacagcgtgctgtacaacagcgccagcttcagcaccttcaagtgctacggcgtgtcccctaccaagctgaacgacctgtgcttcaccaacgtgtacgctgacagcttcgtgatccggggagatgaagtgcggcagattgcccctggacagacaggcaagattgccgactacaactacaagctgcccgacgacttcaccggctgtgtgatcgcctggaatagcaacaacctggacagcaaagtcggcggcaactacaattacctgtaccggctgttccggaagtccaacctgaagcctttcgagcgggacatcagcaccgagatctatcaggccggcagcaccccttgcaatggcgtggaaggcttcaactgctacttcccactgcagtcctacggcttccagcctacaaacggcgtgggctaccagccttacagagtggtggtgctgagcttcgagctgctgcatgctcctgccacagtgtgcggccctaagaaaagcaccaacctggtcaagaacaaatgcgtgaacttc

SEQ ID NO: 16 - protein, 6 aa, Artificial sequence
  Synthetic peptide
GGSGGS

SEQ ID NO: 17 - protein, 10 aa, Artificial Sequence
  Synthetic peptide
GGGGSGGGGS

SEQ ID NO: 18 - protein, 8 aa, Artificial Sequence
  Synthetic peptide
DYKDDDDK